SEQUENCE LISTING (2 sequences)

SEQ ID NO: 1 - DNA, 18 bp, unidentified
  misc_feature 1..18: note = Adapter sequence
  source 1..18: mol_type = other DNA
ggtataggcggaagcgcc

SEQ ID NO: 2 - DNA, 27 bp, unidentified
  source 1..27: mol_type = other DNA
gaacagaaactgattagcgaagaagac

(19) United States
(12) Patent Application Publication
(10) Pub. No.: US 2024/0050549 A1
(43) Pub. Date: Feb. 15, 2024

(54) Recombinant Ranavirus, Methods of Production, and Its Use As A Mammalian Expression System

(71) Applicant: The Trustees of the California State University, Long Beach, CA (US)

(72) Inventors: James Jancovich, San Marcos, CA (US); Nathan Bartlett, Newcastle (AU)

(73) Assignee: The Trustees of the California State University, Long Beach, CA (US)

(21) Appl. No.: 18/488,422

(22) Filed: Oct. 17, 2023

Related U.S. Application Data
(63) Continuation-in-part of application No. 17/212,260, filed on Mar. 25, 2021, now abandoned.
Application No. 17/212,260 is a continuation-in-part of application No. 15/968,241, filed on May 1, 2018, now abandoned.
(60) Provisional application No. 62/500,441, filed on May 2, 2017.

(51) Int. Cl. A61K 39/12; A61P 31/20

(52) U.S. Cl. CPC A61K 39/12; A61P 31/20; A61K 2039/5254

(57) ABSTRACT

A mammalian expression system comprising an attenuated, recombinant Ranavirus that has at least one foreign expression element using a unique combination of mammalian transcriptional and translational enhancement elements is disclosed. In other contemplated embodiments, a mammalian expression system comprising a virus, wherein the virus is engineered to express at least two vaccine antigens is disclosed. In addition, methods of delivering human antigens to a mammal are disclosed that include: providing a non-mammalian virus, engineering a recombinant virus that can express at least one foreign molecule by modifying the non-mammalian virus, and using the recombinant Ranavirus to express and deliver foreign antigens to a mammal.

This United States Continuation In Part Application claims priority to U.S. Utility patent application Ser. No.: 15/968,241 and U.S. Utility patent application Ser. No.: 17/212,260, both of which claim priority to U.S. Provisional Patent Application Ser. No.: 62/500441 entitled “Use of Recombinant Ranavirus as a Human Vaccine Vector” filed on May 2, 2017, which are commonly-owned and incorporated in their entirety by reference.

### FIELD OF THE SUBJECT MATTER

The field of the subject matter is the development of a mammalian expression system to generate anti-viral airway immunity that will prevent various infections.

### INCORPORATION BY REFERENCE OF SEQUENCE LISTING PROVIDED AS A XML FILE

A Sequence Listing is provided herewith as a XML file, “1600142SequenceID2023.xml” created on Oct. 15, 2023 and having a size of 4 KB. The contents of the XML file are incorporated by reference herein in their entirety.

### BACKGROUND

The annual, global cost of respiratory viral infections is in the order of billions of health care dollars. Viruses cause the common cold as well as serious lung conditions such as severe lower respiratory tract viral disease (influenza, respiratory syncytial virus (RSV)) asthma attacks (rhinovirus). School age children are the perfect vector for spread and transmission of respiratory viruses. On average children experience 5-10 colds per year, thus asthmatic kids are particularly susceptible to virus-induced asthma attacks. They are also bringing the virus home from school which can spread colds and can cause an asthma attack in susceptible family members. Indeed, in the USA there is a significant spike in hospital admissions due to asthma attacks in September, which coincides with the start of the school year after the summer break.

According to the Centers for Disease Control (CDC), “common colds are the main reason that children miss school and adults miss work. Each year in the United States, there are millions of cases of the common cold. Adults have an average of 2-3 colds per year, and children have even more. Most people get colds in the winter and spring, but it is possible to get a cold any time of the year. Symptoms usually include sore throat, runny nose, coughing, sneezing, watery eyes, headaches and body aches. Most people recover within about 7-10 days. However, people with weakened immune systems, asthma, or respiratory conditions may develop serious illness, such as pneumonia. The CDC also links rhinovirus infections to sinus and ear infections. In addition, RV infections are highly linked to the development of asthma as well as exacerbate disease in chronic obstruction pulmonary disorder and cystic fibrosis which predisposes individuals to secondary bacterial infections and pneumonia, which can be life threatening. Lung transplant patients are also at risk from respiratory viral infections, also due to secondary bacterial pneumonia. Taken together (100s of subtypes, frequency of infection, ease of transmission by susceptible school-age children, lack of vaccine), it is little wonder that RV are the most common trigger of asthma attacks and infections that can—at the least, impact productivity and at worst—be life-threatening. Prevention of RV infections has real potential to impact on the huge health care burden directly attributable to this virus. Therefore, it would be ideal to find a mammalian expression system to generate antiviral airway immunity that would help combat at respiratory infections.

Viral-vector protein expression platforms are widely used in vaccines and as mammalian expression systems. Most viral-vector protein expression platforms are based on mammalian viruses (e.g. adenovirus, attenuated vaccinia virus). However, this approach is not without safety concerns and can be complicated by pre-existing host immunity to the viral vector. We have developed an alternative approach based on Ambystoma tigrinum virus (ATV; family Iridoviridae, subfamily Alphairidovirnae, genus Ranavirus), a large double-stranded DNA virus that exclusively infects salamanders (cold blooded vertebrates) originally isolated from tiger salamanders (Ambystoma tigrinum) 25 years ago. Since that time, we have extensively characterized the virus and show that the ATV genome can be efficiently manipulated by removing and inserting genetic material. In addition, we have attenuated ATV by deleting non-essential genes and purifying intracellular virions that lack an envelope. ATV has unique replication events in both the nucleus, using cellular expression machinery, and the cytoplasm, using viral specific proteins and we have utilized this unique replication cycle to incorporate at least one mammalian transcription element and at least one translation enhancement element in attenuated ATV that facilitate and enhance protein expression in mammalian cells. Adapted to an amphibian host, ATV does not infect humans therefore pre-existing immunity will not cause complications with using this amphibian- based expression system. In addition, ATV is thermally limited to productive replication below 28° C., therefore it is unable to produce infectious viral particles at temperatures within the human body. However, recombinant, attenuated ATV-expressed recombinant proteins are produced by mammalian (mouse and human) airway epithelial cells at temperatures approaching 37° C. which we seek to exploit to stimulate a protective neutralizing anti-viral IgA response in the airway mucosa.

To demonstrate proof of concept of the utility of the ATV-based protein expression system for vaccine development we show that recombinant ATV delivered via the respiratory route will express protein in primary human bronchial epithelial cells (BECs) differentiated ex vivo at the air-liquid interface (ALI). These data demonstrate ATV-mediated expression of recombinant protein in well differentiated primary human ALI-BECs and is evidence that airway delivery of recombinant, attenuated ATV will express protein in the respiratory mucosa. In addition, our in vivo data suggest that exposure of mice to recombinant, attenuated ATV did not produce signs or symptoms of illness and mouse lungs appeared normal with no overt inflammatory infiltrate yet showed strong expression of the foreign antigen in epithelial cells that line the airway lumen in a temperature sensitive manner. These data suggest that the ATV expression 30 platform will be safe and effective at expressing antigen in the respiratory tract in vivo.

Therefore, we have developed a novel mammalian expression system using a recombinant, attenuated ATV that has been engineered to efficiently and effectively express foreign proteins in mammalian cells without viral replication.

### BRIEF DESCRIPTION OF THE FIGURES

FIG. 1 shows that ATV is a unique Ranavirus strain that forms a monophyletic clade distinct from other Ranaviruses.

FIG. 2 shows a schematic of process for generating a recombinant Ranavirus.

FIG. 3 shows expression of GNR by observing GFP expression in a plaque generated from a recombinant ATV in permissive FHM cells.

FIG. 4 shows ATV temperature sensitivity replication in permissive fathead minnow (FHM) and non-permissive mouse lung epithelial (LA-4) cells.

FIG. 5A shows expression of GNR construct from recombinant ATV in non-permissive mouse lung epithelial cells by fluorescent microscopy. Mouse lung epithelial (LA-4) cells were either mock infected or infected with either wild-type ATV or ATVΔ40L that expresses the GNR construct using the universal cytomegalovirus (CMV) promoter or our unique combination of mammalian transcriptional and translational enhancement elements (TEE) at a multiplicity of infection of 1 or 10 at 31° C.

FIG. 5B shows expression of GNR construct from recombinant ATV in mouse lung epithelial cells. Mouse lung epithelial (LA4) cells were either mock infected or infected with either wild-type ATV or ATVΔ40L that expresses the GNR construct using a CMV promoter or our unique combination of TEE expression elements at a multiplicity of infection of 1 or 10 at 35° C.

FIG. 5C shows expression of GNR construct from recombinant ATV using a CMV promoter or our unique combination of TEE expression elements in mouse lung epithelial cells at 31° C. by western blot analysis.

FIG. 6 shows GNR expression from recombinant ATV in air-liquid interface primary human bronchial epithelial cells. Primary human bronchial epithelial cells were differentiated at the air-liquid interface (BEC-ALI) and either mock infected or infected with 10⁶ pfu/ml of ATVΔ40L that expresses GNR using our unique combination of TEE expression elements at 16 and 40 hpi at 33° C.

FIG. 7 shows GNR expression in mouse trachea and lung tissue. BALB/c mice were either mock infected or infected with 1×10⁶ pfu of recATV that expresses the GNR construct using a CMV promoter or our unique combination of TEE expression elements. Trachea and lungs were obtained 48 hours post infection and histological cross-sections were analyzed for gross morphology and GFP expression.

### SUMMARY OF THE SUBJECT MATTER

A mammalian expression system comprising an attenuated, recombinant Ranavirus that has at least one foreign expression element is disclosed. In other contemplated embodiments, a mammalian expression system comprising a virus, wherein the virus is engineered to express at least two vaccine antigens is disclosed.

In addition, methods of delivering human antigens to a mammal are disclosed that include: providing a non-mammalian virus, engineering a recombinant virus that can express at least one foreign molecule by modifying the non-mammalian virus, and using the recombinant Ranavirus to deliver human antigens to a mammal.

### DETAILED DESCRIPTION

In view of earlier-presented information, the ideal vector for a human antigen expression system for generation of antiviral airway immunity is a large DNA virus that can be engineered to express one or multiple foreign antigens. Importantly, this virus should not productively infect human cells. Instead, it needs to enter human cells, express antigens but not form a new virus, which is called abortive replication. The best place to find such a viral vaccine vector is to look in animals that are very distantly related to humans.

Specifically, a contemplated mammalian expression system for generating antiviral airway immunity comprises an attenuated, recombinant Ranavirus that has at least one foreign expression element. Contemplated recombinant, attenuated viruses are unique in that they have been deleted of pathogenesis genes and viral envelope and those genes are replaced with expression constructs, for example and including mammalian promoter elements driving expression of at least one antigen.

As used herein, the term “attenuated” with respect to a virus or viral mammalian expression vector means a virus platform created by reducing the virulence of a pathogen, but still keeping it viable (or “live”). Attenuation takes an infectious agent and alters it so that it becomes harmless or less virulent. These vaccines are in contrast to those produced by “killing” the virus (inactivated vaccine). An attenuated virus may be used as a mammalian expression vector that is capable of expressing foreign antigens thus stimulating an immune response and creating immunity in a patient, but not of causing illness in that same patient. In contemplated embodiments, viruses have been deleted of pathogenesis genes and viral envelope. There are currently 4 loci/genes in the contemplated virus that can be deleted and foreign material inserted and in contemplated embodiments we include data for one locus; however, in other contemplated embodiments, other loci or genes or numbers of loci or genes can be deleted and foreign material inserted. In some contemplated embodiments, in place of the pathogenesis gene(s), a mammalian virus promoter element and a human translation enhancement element have been inserted that drive expression of a foreign antigen.

In contemplated embodiments, the at least one foreign expression element expresses at least one foreign protein, at least two foreign antigens, at least one virus-like particle or a combination thereof.

In some contemplated embodiments, a mammalian expression system comprises an attenuated virus, wherein the virus is engineered to express at least two vaccine antigens. In some of these contemplated embodiments, the virus is an attenuated recombinant ATV.

In addition, methods of delivering human antigens to a mammal are disclosed that include: providing a non-mammalian virus, engineering a recombinant virus that can express at least one foreign molecule by modifying the non-mammalian virus with a unique combination of transcription and translation enhancement elements, and using the recombinant Ranavirus to express and deliver human antigens to a mammal.

Conventionally, and as shown in Aron et al. (2017), engineering a recombinant virus includes: generating a recombination cassette, wherein the cassette contains homologous sequences flanking a screenable and selectable reporter gene driven by a promoter, infecting at least one cell with the attenuated non-mammalian virus, transfecting the at least one cell with the recombination cassette to form a combination of the at least one cell and the wild-type non-mammalian virus, harvesting a modified combination of the at least one cell and the attenuated non-mammalian virus; and selecting from the modified combination the recombinant virus deleted of the target open reading frame or ORF by serial passaging in cells treated with selection specific components.

As disclosed herein, contemplated vaccine vectors can be used to reduce the occurrence of mammalian respiratory disease and/or related diseases or conditions.

All animals, including cold-blooded amphibians, are host to a variety of viruses, including salamanders. Salamander models have been used in other research related to human conditions. For example, Del Priore et al. looked at salamander research to find a connection between retinal cell apoptosis and increasing age. (Lucian V. Del Priore, Ya-Hui Kuo and Tongalp H. Tezel, “Age-Related Changes in Human RPE Cell Density and Apoptosis Proportion In Situ”, Investigative Ophthalmology & Visual Science, October 2002, Vol. 43, 3312-3318 citing Townes-Anderson E, Colantonio A, St Jules R S. “Age-related Changes in the Tiger Salamander Retina”, Exp Eye Res. 1998;66:653-667). Wagner et al. used fish models, including aquatic salamanders to show that there is evidence of a stanniocalcin-like hormone in humans, specifically human kidneys. (Graham R. Wagern, Collete C. Guiraudon, Christine Milliken and D. Harold Copp,“Immunological and Biological Evidence for a Stanniocalcin-like Hormone in Human Kidney”, Proc. Natl. Acad. Sci. USA, 92 (1995).

As a basis for this research, Arizona salamanders were captured and, upon investigation, showed signs of illness. After significant examination and analysis, a new virus, now called Ambystoma tigrinum virus (ATV), was found. This virus is a member of the genus Ranavirus, subfamily Alphairidoviridae, family Iridoviridae—the members of which are large DNA viruses that infect insects, amphibians, reptiles and fish.

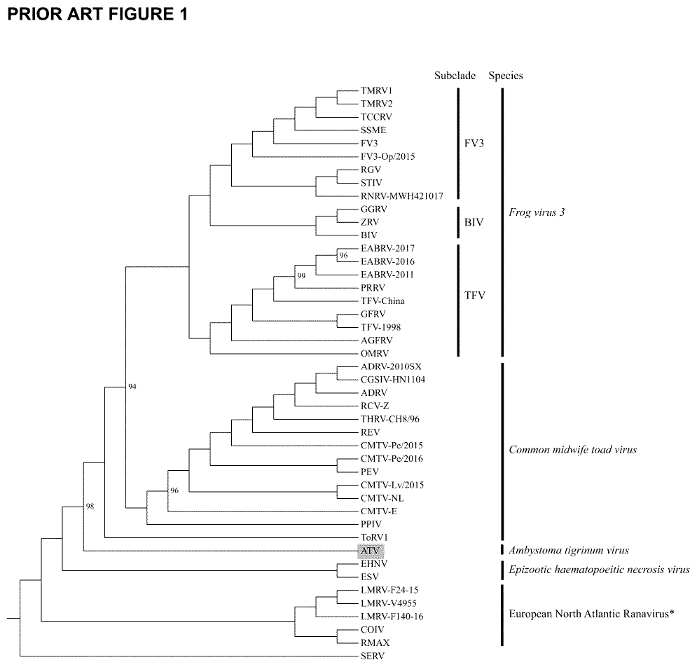

ATV is a unique Ranavirus strain that forms a monophyletic clade distinct from other viruses in this genus when comparing the 26 core iridovirus genes (FIG. 1). Wild-type (wt) ATV encodes 90 proteins and is host restricted to salamanders, unlike ranaviruses Frog virus 3 (FV3) of Bohle iridovirus (BIV) that are promiscuous pathogens and infect multiple host species. In addition, ATV has a unique overall gene order as compared to other ranaviruses. While the ATV gene order is similar to fish viruses from Australia and Europe (EHNV and ESV, respectively) these fish ranaviruses are larger in size (˜127,000 bp) as compared to ATV (˜106,000 bp) and encode around 100 proteins.

Since this discovery, the researchers spent several years identifying and characterizing non-essential putative pathogenesis genes and perfecting the technique for making attenuated recombinant ATVs (recATV) that efficiently expresses antigens in non-permissive cells. In addition, the researchers have identified the viral envelope as a pathogenesis factor and have optimized foreign gene expression in ATV by insertion of a unique combination of mammalian expression components into identified non-essential gene loci. For example, a recATV was created that expresses two proteins fused together: a green fluorescent protein (GFP) fused to a neomycin resistance gene (NR) that causes the virus to be resistant to neomycin treatment and infected cells to glow green. The GFP-NR fusion construct, referred to as GNR, is expressed by incorporating into recATV a unique combination of mammalian transcription and translation enhancement elements that effectively expresses GNR in non-permissive cells in vitro, in differentiated primary human cells ex vivo and in mouse lungs and trachea in vivo without viral replication. In addition, a mouse model system was developed for ATV infections and test compounds, and other agents to fight disease, are routinely tested in this model system.

The new recATV will be utilized, as disclosed herein with the unique combination of mammalian transcription and translation enhancement elements, in mouse studies to prove that it can function as a mammalian expression system. The recATV mammalian expression system will be used as a vector to deliver and express protective antigens from mammalian pathogens. FIGS. 1-7 show some of the preliminary results and information related to this invention.

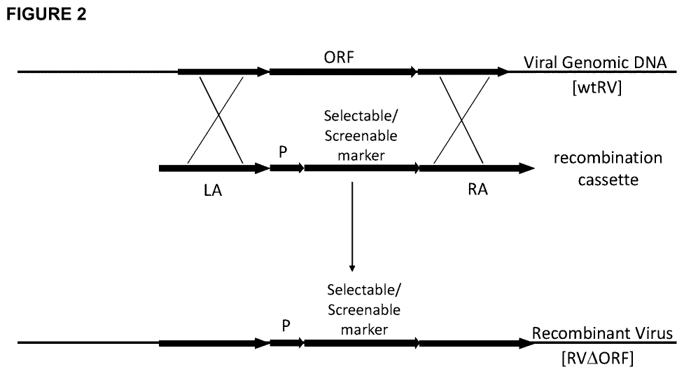

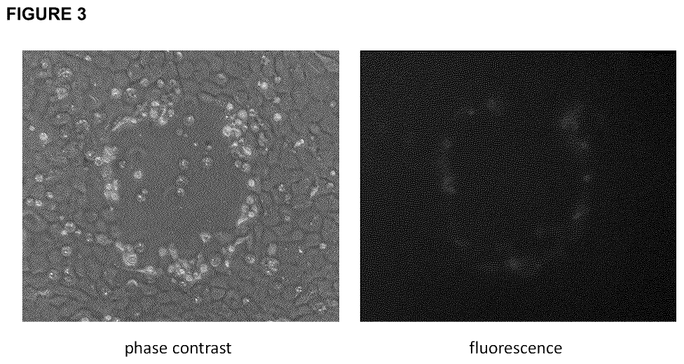

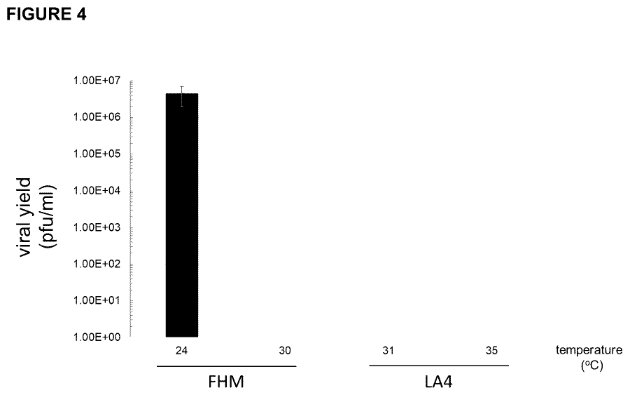

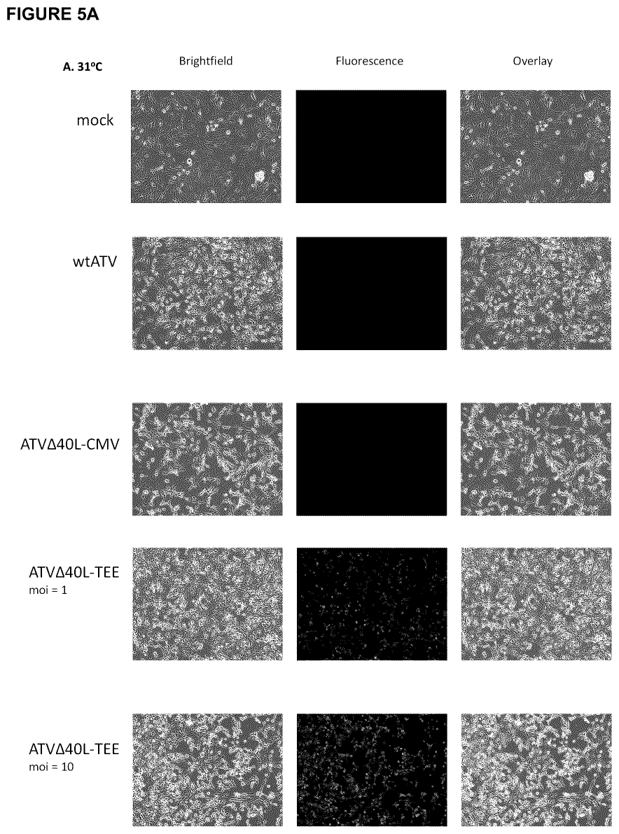

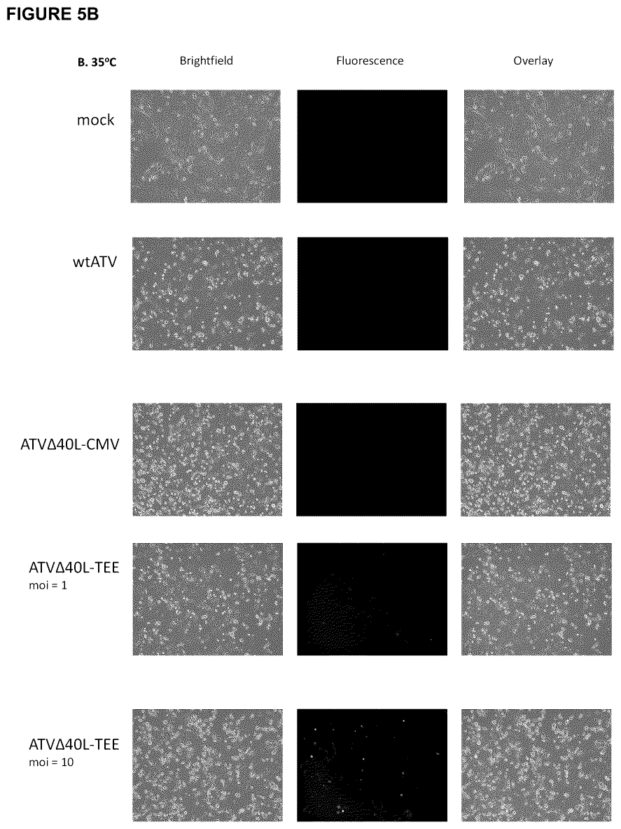

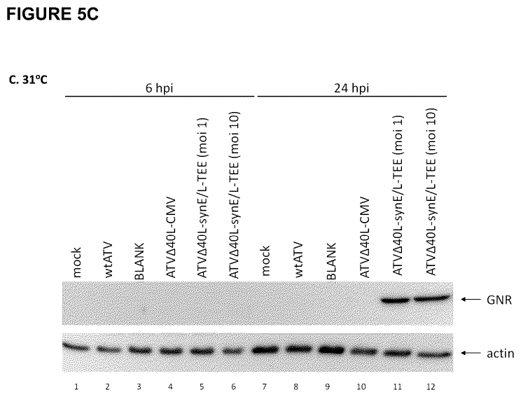

Specifically, the data show that ATV is a unique Ranavirus within the genus Ranavirus (FIG. 1). While ATV shares gene sequence homology with other ranaviruses, ATV is a thermally limited to replication below 28° C. and host restricted pathogen compared to other members of the genus. Therefore, a mutant, attenuated ATV expressing two proteins fused together, the green fluorescent protein (GFP) that is fused to a selectable marker, neomycin resistance (NR), collectively referred to as GNR (FIG. 2) that is expressed using a unique combination of mammalian transcription and translation enhancement elements (recATV-TEE) was developed. We show that GNR is efficiently expressed in fish cells that are susceptible to ATV (FIG. 3). RecATV-TEE is temperature sensitive and does not produce infectious viral particles in non-permissive cells (i.e. LA-4) but does replicate in permissive cells (i.e. FHM) in a temperature sensitive manner (FIG. 4). Since the recATV mammalian expression system contemplated herein is designed to express antigens in mammalian cells without replication while generating antiviral airway immunity for mammalian respiratory diseases, it has been shown that expression of the GNR construct in mouse lung epithelial cells using the TEE is significantly enhanced as compared to a well characterized, and routinely used cytomegalovirus (CMV) promoter (FIG. 5; recATV-CMV). Expression of GNR from recATV-TEE is temperature sensitive with reduced expression at 35° C. as compared to 31° C. and no expression was observed at 37° C. (data not shown). Collectively, these data show our unique mammalian expression system is efficient at expressing GNR in vitro in non-permissive cells without replication.

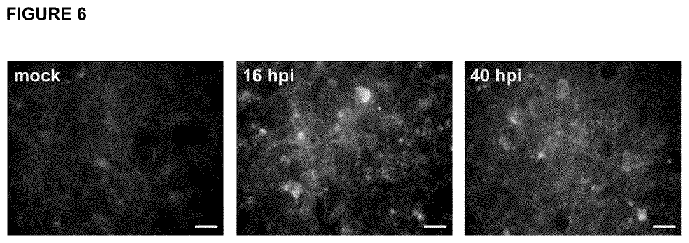

We have used the recATV mammalian expression system platform to demonstrate GNR expression ex vivo using primary human bronchial epithelial cells (BECs) differentiated for 28 days at the air-liquid interface (ALI) to generate mucus producing and ciliated stratified epithelial cell cultures (FIG. 6). ALI-BECs were mock treated or treated with recATV-TEEat 33° C. GNR expression was detected by observing GFP expression in ALI-BECs by 16 h and continued through 40 h post-apical treatment with recATV-TEE. These data demonstrate ATV-mediated expression of recombinant protein in well differentiated primary human ALI-BECs and is evidence that airway delivery of recombinant ATV will express protein in the respiratory mucosa.

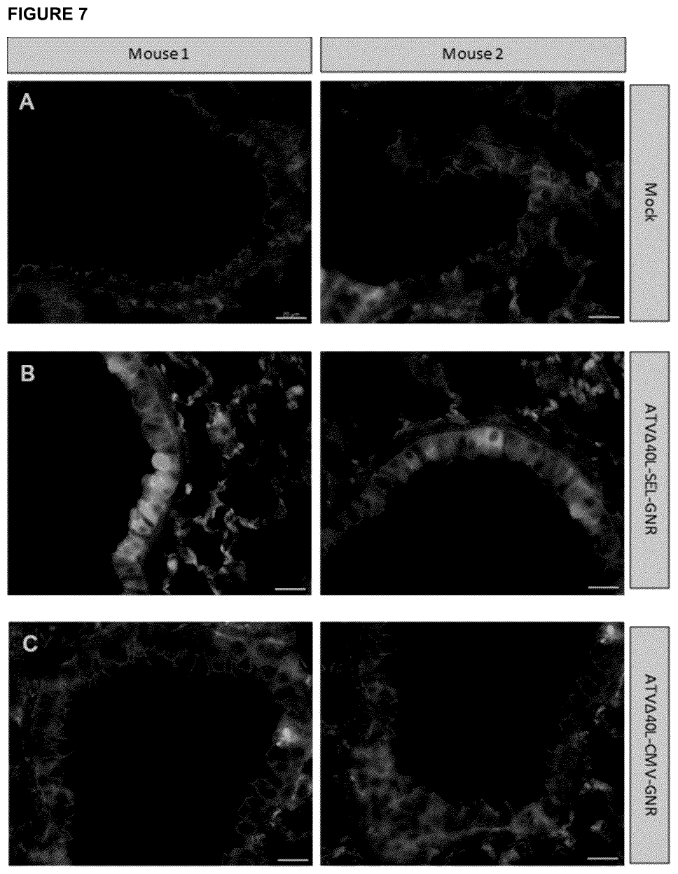

We have used recATV to confirm safety and recombinant protein expression in the airways of BALB/C mice. Mice were inoculated i.n. with 1×10⁶ PFU of recATV-TEE or recATV-CMV under light isoflurane anesthesia. Control mice were dosed with the same volume of PBS. All ATV-treated mice (n=4, recATV-TEE and recATV-CMV) showed no signs of illness at any time during the experiment. Trachea and lungs were obtained after 48 hours and histological cross-sections were analyzed for gross morphology and GFP expression. Lung histological features of mice infected with recATV appeared normal with no overt inflammatory infiltrate (data not shown). Low level background fluorescence was apparent in PBS-treated mice (FIG. 7) whereas strong GFP expression, predominantly in epithelial cells that line the airway lumen, was observed in recATV-TEE treated mice (FIG. 7) at levels above mice treated with recATV-CMV. These data suggest that the recATV expression platform will be safe and effective at expressing foreign antigens in the respiratory tract in vivo.

Collectively, the data suggest a novel antigen expression system that can be used to develop protective airway immunity for mammalian (i.e. human) respiratory disease has been developed. Each of these figures will be described in detail below.

FIG. 1 shows a cladogram depicting the relationship of the Thai TFVs to other members of the genus Ranavirus based on the concatenated locally collinear blocks alignments. All nodes are supported by bootstrap values of 100% from the Maximum Likelihood analysis except the nodes labelled with bootstrap values. See Tables 1 and 2 for viral abbreviations. *Note: European North Atlantic Ranavirus has not been approved as a ranaviral species by the International Committee on Taxonomy of Viruses. From Sriwanayos P, Subramaniam K, Stilwell N K, Imnoi K, Popov V L, Kanchanakhan S, Polchana J, and Waltzek T B. 2020. Phylogenomic characterization of ranaviruses isolated from cultured fish and amphibians in Thailand. FACETS 5: 963-979.doi:10.1139/facets-2020-0043.

FIG. 2 shows a schematic of process for generating a recombinant Ranavirus. The process of generating a knock-out Ranavirus (RV) deleted of the target gene requires the generation of a recombination cassette that contains homologous sequences (LA and RA) flanking a screenable and selectable reporter gene driven by a promoter (P). Cells are infected with wild-type virus and then transfected with the recombination cassette. Cells and virus are harvested after 48 hours and the recombinant virus deleted of the target ORF is selected by serial passaging in cells treated with selection specific components. Recombinant virus deleted of the target ORF will be resistant to the selection substance and produce easily observable plaques.

FIG. 3 shows expression of GNR by observing GFP expression in a plaque generated from a recombinant ATV in permissive FHM cells. ATV mutant virus plaque under phase contrast and fluorescent microscopy.

FIG. 4 shows ATV temperature sensitivity replication in permissive fathead minnow (FHM) and non-permissive mouse lung epithelial (LA-4) cells. FHM or LA4 cells were infected with recATV-TEE (i.e. ATVΔ40L-GFP) at a multiplicity of infection (MOI) of 0.01 pfu/cell. After the 1 hour of infecting cells, the inoculum was removed and the cells were overlayed with growth medium. Cells and virus were harvested at 72 hours post infection and assayed for viral growth by plaque assay in FHM cells. Viral yield was determined by calculating the amount of virus produced from the amount of virus used to infect cells.

FIGS. 5A-C shows temperature sensitive expression of GNR construct from recombinant, attenuated ATV in mouse lung epithelial cells. Mouse lung epithelial (LA-4) cells were either mock infected or infected with either wild-type ATV or recATV that expresses the GNR construct using a well characterized cytomegalovirus (CMV) promoter or our unique combination of mammalian transcription and translation enhancement elements (TEE) (i.e. ATVΔ40L-CMV and ATVΔ40L-TEE, respectively) at a multiplicity of infection of 1 or 10 at 31° C., 35° C. or 37° C. Cells were) analyzed by florescent microscopy for GFP expression (panels A and B) or harvested at the indicated time points and total proteins were isolated before analysis for GNR expression by Western blot (panel C). Data for the 37° C. are not shown as not GFP expression was not observed at this temperature. FIG. 5A shows expression of GNR construct from recombinant ATV in mouse lung epithelial cells by observing GFP expression by fluorescent microscopy at 31° C. FIG. 5B shows expression of GNR construct from recombinant ATV in mouse lung epithelial cells by observing GPF expression by fluorescent microscopy 35° C. FIG. 5C shows expression of GNR construct from recombinant ATV in mouse lung epithelial cells at 31° C. by western blot analysis. All data show increased expression from recATV-TEE (i.e. ATVΔ40L-TEE) as compared to recATV-CMV (i.e. ATVΔ40L-CMV) suggesting that our unique combination of mammalian transcription and translation enhancement elements significantly enhances GNR expression over conventional viral promoters (i.e. CMV).

FIG. 6 shows ex vivo GNR expression from recATV in air-liquid interface (ALI)-differentiated primary human bronchial epithelial cells (BECs). ALI-BECs were mock treated or treated with recATV-TEE (i.e. ATVΔ40L-GFP) at 33° C. Cells were fixed and stained at 16 and 40 hpi with junction marker ZO-1 shown in red and GFP in green. Cell nuclei stained with DAPI are shown in blue. GFP expression was observed in ALI-BECs by 16 hpi and continued through 40 hpi infected with our vaccine platform virus, ATVΔ40L-GFP, and GFP expression was not observed in mock treated cells. These data demonstrate the inherent in vitro temperature sensitivity (i.e. safety) of the ATV system and confirms ATV expression of foreign genes in mammalian cells at human airway temperatures (i.e. 31-35° C.) without viral replication.

FIG. 7 shows GNR expression from recATV in vivo. Wild type BALB/c mice were intranasally inoculated with mock (PBS) (A), or 1×10⁶ PFU of recATV-TEE (i.e. ATVΔ40L-SEL-GNR) (B) or recATV-CMV (i.e. ATVΔ40L-CMV-GNR) (C) under light isoflurane. Immunofluorescence was performed on histological cross-sections of formalin-fixed paraffin embedded lung tissue at 48 hours post-infection. Images show transverse sections at 63× magnification where red reflects ZO-1 staining, blue reflects DAPI counterstain and green is GFP (translated protein from genetically modified ATV strains).

### MATERIALS AND METHODS

The following materials and methods were used to obtain and collect the data presented herein.

### Cells and Virus

Fathead minnow (FHM; ATCC CCL-42) cells were maintained in Minimum Essential Medium with Hank Salts (HMEM) (Gibco) supplemented with 5% fetal bovine serum (FBS) (Hyclone) and 0.1 mM nonessential amino acids and vitamins (Invitrogen). FHM cells were incubated at 20 to 22° C. in the presence of 5% CO₂. LA-4 mouse lung epithelial cells (kindly provided by Dr. Bianca Mothé and the La Jolla Institute of Allergy and Immunology) were maintained in F12K medium supplemented with 15% FBS and incubated at 37° C. with 5% CO₂. Wild-type Ambystoma tigrinum virus (wtATV), was originally isolated from tiger salamanders in Southern Arizona (Jancovich et al., 1997). Wild-type and mutant ATV were amplified and quantified in FHM cells. Briefly, viral amplification was performed in 100 mm dishes of FHM cells that were infected with virus at a multiplicity of infection of 0.01, rocked for 1 hr and then overlayed with HMEM with 5% FBS. Infected cells were monitored for cytopathic effects (CPE). Once CPE reached 95-100%, infected cells were harvested, concentrated by centrifugation at 1,000×g for 10 min and the pellet of infected cells resuspended in 100 μl of 10 mM Tris, pH 8.0. Virus was released by 3 cycles of freeze/thaw followed by centrifugation at 1,000×g for 10 min to clarify cellular debris. The supernatant containing virus was quantified by plaque assay in FHM cells.

### Generating Recombination Cassettes

Recombination cassettes to delete a target gene, or open reading frame (ORF) and insert a foreign antigen were generated by designing forward (for) and reverse (rev) primers to amplify the upstream (LA) and downstream (RA) flanking sequences of the gene to be deleted. Primers were designed to initially amplify a PCR product around 1,000 nt up- and downstream from the start and end of the target sequence, respectively. These primers (ORF#_LA_for_1k and ORF#_RA_rev_1k, respectively) were paired with primers designed immediately before the start (ORF#_LA_rev) and after the end (ORF#_RA_rev) of the target gene. An adapter sequence (AF; 5′ GGTATAGGCGGAAGCGCC 3′) was added to the 3′ end of the LA reverse primer (AF_ORF#_LA_rev) and a second adapter (AR; 5′ GAACAGAAACTGATTAGCGAAGAAGAC 3′) was added to the 5′ end of the RA forward primer (AR_ORF#_RA_for). Each of these primers were designed to have a predicted melting temperature around 60° C. Pairing the ORF#_LA_for_1k primer with the AF_ORF#_LA_rev and the AR_ORF#_RA_for with ORF#_RA_ 1k_rev generated approximately 1 kb of sequence of both the left and right flanking homologous sequences with adapters at the 3′ end of the LA and the 5′ end of the RA.

Primers are then designed to amplify promoter regions and a screenable and selectable marker that will allow for the construction of recATV deleted of a specific ORF. The screenable and selectable marker includes a green fluorescent protein (GFP) fused to a neomycin resistant gene (NR) gene, herein referred to as GNR (Hansen, S. G., Cope, T. A., Hruby, D. E. 2002. BiZyme: A novel fusion protein-mediated selection of vaccinia virus recombinants by fluorescence and antibiotic resistance. BioTechniques 32:1178-1187). The GNR forward primer (AF-CMV-GNR for OR AF-p-TEE-GNR for) includes a 5′ adapter (AF) followed by a specific promoter region: (i) a cytomegalovirus (CMV) promoter (Thomsen, D. R., Stenberg, R. M., Goins, W. F., Stinski, M. F. 1984. Promoter-regulatory region of the major immediate early gene of human cytomegalovirus. PNAS 81: 659-663; Foecking, M. K., Hofstetter, H. 1986. Powerful and versatile enhancer-promoter unit for mammalian expression vectors. Gene 45:101-105.) OR (ii) a vaccinia virus synthetic early-late promoter (Chakrabarti, S., Sisler, J. R., Moss, B. 1997. Compact, synthetic, vaccinia virus early/late promoter for protein expression. BioTechniques 23:1094-1097) combined with a human translation enhancement element (TEE; Wellensiek B P, Larsen A C, Stephens B, Kukurba K, Waern K, Briones N, Liu L, Snyder M, Jacobs B L, Kumar S, Chaput J C. 2013. Genome-wide profiling of human cap-independent translation-enhancing elements. Nat Methods 10:747-50). The GNR reverse primer (AR-GNR rev) includes the adapter reverse (AR). Using primers AF-CMV-GNR for or AF-p-TEE-GNR for and AR-GNR rev and a pcDNA3.1 vector containing the GNR construct as a template the pGNR product is generated. For each PCR reaction, 50 ng of plasmid or 100 ng of viral DNA was added to the High Fidelity PCR Master Mix according to the manufacturer's instructions (Roche) and DNA was amplified with a single cycle of 94° C. for 2 minutes, followed by 25 cycles of 94° C. (30 seconds), 50° C. (for primer sets seq for/rev and 500_for/rev) or 55° C. (for primer set 1k_for/rev) (30 seconds), 72° C. (90 seconds) and a final cycle of 72° C. for 7 minutes. PCR products were visualized by 1% agarose gel electrophoresis and products were purified by Wizard® SV Gel and PCR Clean-Up System (Promega) system as described by the manufacturer after excision from 0.7% agarose gel. Purified PCR products were quantified by Nanodrop spectrophotometry. At this point we have three purified PCR products for each ATV ORF: the LA, RA and pGNR. The adapter at the 3′ end of LA corresponds to the adapter at the 5′ end of pGNR while the adapter at the 3′ end of pGNR corresponds to the adapter at the 5′ end of the RA. These adapters allow for overlapping PCR to combine the three PCR products (i.e. LA, pGNR and RA) using a standardized protocol.

To generate a recombination cassette by overlapping PCR, 50 ng of each PCR product (LA, RA and pGNR) was added to 45 μl reaction (final volume) containing 1×iProof HF buffer, 200 μM of each dNTP, and 0.02 U/μl iProof DNA polymerase (BioRad). The recombination cassette assembly was initiated by a single cycle of 98° C. (30 seconds), followed by 7 cycles of 98° C. (10 seconds), 58° C. (28 minutes), 72° C. (150 seconds). After the completion of this program, 0.5 μM of the ORF#_LA_1k_for and ORF#_RA_1k_rev were added along with another 0.02 U/μl iProof DNA polymerase. The reaction was then returned to the thermocycler and a second program consisting of a single cycle of 98° C. (30 seconds), followed by 35 cycles of 98° C. (10 seconds), 55° C. (30 seconds), 72° C. (150 seconds) and a final cycle of 72° C. for 5 minutes was performed. PCR products were visualized and purified as described above. Purified recombination cassettes were then re-amplified using the ORF#_LA_500_for and ORF#_RA_500_rev primers using the High Fidelity PCR Master Mix as described above. PCR products were visualized and purified as described above and then cloned into pCR2.1®-TOPO® cloning vector as per the manufacturer's instructions (Thermo Fisher Scientific). Colonies were screened for the recombination cassette using the seq for/rev primer set for each ORF and correctly constructed recombination cassettes were confirmed by sequencing. The recombination cassette was PCR amplified from the plasmid, agarose gel purified and quantified as described above for use in generating a knockout virus.

### Generating Knockout ATV

Approximately 50% confluent monolayers of FHM cells in 35 mm dishes were infected with wtATV at a MOI of 0.01 for 1 hour at room temperature. While the virus was attaching, 500 ng of the target ATV ORF recombination cassette that had been PCR amplified and purified was added to FuGene° 6 transfection reagent according to the manufacturer's instructions (Promega). This solution was incubated at room temperature for 20 minutes. After 1 hour, the virus inoculum was removed and replaced with the DNA-FuGene® 6 mixture. Cells were rocked with the transfection mixture for 1 hour at room temperature. After rocking, the infected/transfected cells were overlayed with 1×HMEM medium containing 5% FBS and incubated for 48 hours. Infections were then harvested and subjected to three rounds of freeze-thaw to release virus from the cell. The sample was then clarified by centrifugation at 1,000×g for 10 minutes and recombinant viruses were selected by multiple blind passages in confluent monolayers of FHM cells in the presence of 1 mg/mL G418 (i.e. neomycin). wtATV, which is sensitive to G418, was used as a control. The presence of a GFP expressing, neomycin resistant virus plaque was indicative of the generation of a recombinant ATV with a knock-out of the target gene. GFP-neomycin resistant virus was then plaque purified up to four times in the presence of 1 mg/ml G418, grown to high titers as described above and viral DNA was isolated as previously described (Jancovich and Jacobs, 2011). PCR confirmation of the ORF knock-out virus and sequencing around the ATV gene of interest was performed using the seq for/rev primer pair described above. We generated two viruses that express GNR from the ATV 40L locus each using different promoter regions: (i) a CMV promoter (ATVΔ40L-CMV-GNR) and (ii) our unique combination of mammalian transcriptional and translational enhancement elements (ATVΔ40L-SEL-GNR).

### RT-PCR Analysis of Cellular Gene Expression

Total RNA from infected cells was extracted using Qiashredder columns followed by RNA isolation using the RNeasy kit as described by the manufacturer (Qiagen). RNA was quantified by spectrophotometric analysis and cDNA was synthesized from 1 μg of total RNA using random primers and the SuperScript® III Reverse Transcriptase (Invitrogen Life Technologies) as directed by the manufacture. Amplification of specific genes, including GNR, was performed. PCR reactions (50 μl) were performed using the High Fidelity Taq Polymerase Master Mix kit (Roche Diagnostics). Reactions were incubated at 94° C. for 2 minutes followed by 25 cycles of 94° C. for 30 seconds, 55° C. for 30 seconds, and 72° C. for 90 seconds, and a final elongations cycle of 72° C. for 7 minutes. Amplified products were separated on a 1% agrose gel electrophoresis and visualized using a G:Box imaging platform (Syngene).

### Cell Extractions and Western Blot Analysis

Infected cell lysates were collected in 1×SDS sample buffer (50 mM Tris, pH 6.8; 2% SDS; 0.1% bromophenol blue; 10% glycerol; 100 mM betamercaptoethanol) before purification by Qiashredder collection column (Qiagen). Equal cell volumes of cellular extracts were subjected to SDS-PAGE on 12% polyacrylamide gels. Proteins were transferred to either a nitrocellulose membrane or a PVDF membrane at 100 volts for 60 minutes in 10 mM CAPS, pH 11.0, with 20% methanol and 14 mM 2-mercaptoethanal. The blot was blocked for 1 hour in 1×TBS with milk (20 mM Tris-HCl [pH 7.8]; 180 mM NaCl; 3% nonfat dry milk). The blots were incubated overnight at 4° C. with primary antibodies at the appropriate dilution as outlined by the manufacturer (Abcam). Primary antibodies were removed, and the blot was washed three times with 1×TBS containing milk for 30 minutes at room temperature. The blot was then probed with a 1:15,000 dilution of goat anti-rabbit or rabbit anti-mouse IgG-peroxidase conjugate antibody (Sigma) for 1 hour at room temperature. These secondary antibodies were then removed, and the blot was washed three times for 10 minutes each in 1×TBS with milk and then washed three times for 5 minutes each in 1×TBS without milk. The blot was visualized after treatment with the Super Signal Dura chemiluminescent kit according to the manufacturer's instructions using the G:Box imaging platform (Syngene). The relative intensity of proteins was quantified using the GeneTools analysis software (Syngene).

### RecATV Expression ex vivo in Air-Liquid Interface (ALI)-Differentiated Primary Human Bronchial Epithelial Cells (BECs)

Primary human bronchial epitheliam cells (BECs) were revived and expanded in T75 flasks from liquid nitrogen vials using BEGM media (Lonza, Switzerland). Following cell expansion, BECs were trypsinised and seeded in transwell inserts (Corning, United States; 2×10⁵ cells per insert) in ALI initial media comprised of bronchial epithelial base medium and Dulbecco's modified eagle medium (BEBM:DMEM; 50:50 ratio) containing hydrocortisone (0.1%), bovine insulin (0.1%), epinephrine (0.1%), transferrin (0.1%), bovine pituitary extract (0.4%) and ethanolamine (80 μM), MgCl₂ (0.3 mM), MgSO₄ (0.4 mM), bovine serum albumin (0.5 mg/mL), amphotericin B (250 μg/mL), all-trans retinoic acid (30 ng/ml), penicillin/streptomycin (2%), and recombinant human epithelial growth factor (rhEGF) (10 ng/ml) for 3-5 days until confluent. Once confluent, apical media was removed (day 0 of ALI). Basal media was changed on alternative days with ALI final media, containing lower rhEGF concentrations (0.5 ng/mL).

ALI cultures were mock infected or infected apically with 1×10⁶ PFU of recATV-TEE (i.e. ATVΔ40L-GFP). The inoculum was added to the apical surface of cultures for 6 h in 250 μL BEBM with supplements, 1% Insulin-Transferrin-Selenium (ITS) and 0.5% Linoleic Acid (LA). Infection media was then replaced with 500 μL fresh BEBM (with supplements) for the remainder of the experiment. The cells were fixed in 10% neutral-buffered formalin and stained at 16 and 40 hpi with junction marker ZO-1 shown in red and GFP in green. Cell nuclei stained with DAPI are shown in blue. Scale bar 20 μm. Overlay images are presented.

### Mouse Safety Trial

BALB/C mice were infected intranasally with 1×10⁶ PFU of recATV that expresses GNR using the CMV promoter (i.e. ATVΔ40L-CMV-GNR) or our unique combination of mammalian transcriptional and translational enhancement elements (i.e. ATVΔ40L-SEL-GNR) under light isoflurane. Control mice were inoculated with the same volume of PBS. Infected mice were monitored for disease symptoms throughout the course of the experiment. Trachea and lungs were obtained 48 hours post infection and histological cross-sections were analyzed for gross morphology and GFP expression. Lung histological features of mice infected with ATVΔ40L-GFP appeared normal and no anomalies were identified. FP expression was observed at background levels in control mice. However, GFP expression was observed in the upper and middle airway epithelium. These data are consistent with the thermal limitation of recATV and correlate with the LA-4 in vitro and ALI-BEC ex vivo data. Therefore, recATV is safe and effective at expressing GFP in vivo.

Thus, specific embodiments of a recombinant Ranavirus, along with methods of use of contemplated recombinant Ranavirus as a mammalian expression system have been disclosed. It should be apparent, however, to those skilled in the art that many more modifications besides those already described are possible without departing from the inventive concepts herein. The inventive subject matter, therefore, is not to be restricted except in the spirit of the disclosure herein. Moreover, in interpreting the specification, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

TABLE 1

| | | | Naked | Naked | Enveloped | Enveloped |
|---|---|---|---|---|---|---|
| | | | virion | virion | virion | virion |
| | | | apex-apex | side-side | apex-apex | side-side |
| Viral name | | | (nm), | (nm), | (nm), | (nm), |
| (abbreviation) | Isolate | Host | mean (SD) | mean (SD) | mean (SD) | mean (SD) |
| Tiger frog virus | AV9803 | Tiger frog | 157.3 (2.1) | 132.5 (3.2) | 196.5 (4.9) | 187.3 (5.7) |
| (TFV-1998) | | (Hoplobatrachus | | | | |
| | | tigerinus) | | | | |
| Oxyeleotris | D2008 | Marbled sleeper | 158.8 (1.2) | 127.6 (1.6) | 201.3 (2.8) | 186.3 (7.6) |
| marmorata | | goby (Oxyeleotris | | | | |
| ranavirus | | marmorata) | | | | |
| (OMRV) | | | | | | |
| Poecilia | F2112 | Guppy (Poecilia | 158.2 (1.6) | 123.5 (2.3) | 203.7 (4.0) | 187.3 (3.7) |
| reticulata | | reticulata) | | | | |
| ranavirus | | | | | | |
| (PPRV) | | | | | | |
| Goldfish | F0207 | Goldfish | 150.2 (1.2) | 125.5 (1.6) | 185.5 (1.5) | 164.3 (3.1) |
| ranavirus | | (Carassius | | | | |
| (GFRV) | | auratus) | | | | |
| Asian grass frog | D03- | Asian grass frog | 158.7 (1.0) | 128.9 (1.4) | 201.7 (2.1) | 185.3 (3.8) |
| ranavirus | 034 | (Fejervarya | | | | |
| (AGFRV) | | limnocharis) | | | | |
| East Asian | D11- | East Asian | 158.8 (1.3) | 130.4 (1.6) | 194.9 (4.5) | 177.6 (5.6) |
| bullfrog | 067 | bullfrog (H. | | | | |
| ranavirus | | rugulosus) | | | | |
| (EABRV-2011) | | | | | | |
| East Asian | VD-16- | East Asian | 158.9 (1.7) | 129.8 (2.7) | 206.0 (3.3) | 185.8 (4.0) |
| bullfrog | 006 | bullfrog (H. | | | | |
| ranavirus | | rugulosus) | | | | |
| (EABRV-2016) | | | | | | |
| East Asian | VD-17- | East Asian | NO | NO | NO | NO |
| bullfrog | 007 | bullfrog (H. | | | | |
| ranavirus | | rugulosus) | | | | |
| (EABRV-2017) | | | | | | |

Note
Means (±standard deviation, SD) are based on the measurement of 20 unenveloped virions and 3-16 enveloped virions per isolate.
NO, not observed.

TABLE 2

| | Viral | GenBank |
|---|---|---|
| Viral name | abbreviation | Accession No. |
| Frog virus 3 | FV3 | AY548484 |
| Tiger frog virus | TFV | AF389451 |
| Rana grylio iridovirus | RGV | JQ654586 |
| Soft-shelled turtle iridovirus | STIV | EU627010 |
| Bohle iridovirus | BIV | KX185156 |
| German gecko ranavirus | GGRV | KP266742 |
| Ambystoma tigrinum virus | ATV | AY150217 |
| Epizootic haematopoietic necrosis | EHNV | FJ433873 |
| virus | | |
| European sheatfish virus | ESV | JQ724856 |
| Common midwife toad virus | CMTV-E | JQ231222 |
| Common midwife toad virus | CMTV-NL | KP056312 |
| Testudo hermanni ranavirus | THRV-CH8/96 | KP266741 |
| Tortoise ranavirus isolate 1 | ToRV1 | KP266743 |
| Frog virus 3 isolate SSME | SSME | KJ175144 |
| Andrias davidianus ranavirus | ADRV | KC865735 |
| European catfish virus | ECV | KT989885 |
| Short-finned eel ranavirus | SERV | KX353311 |
| Ranavirus maximus | Rmax | KX574343 |
| Cod iridovirus | CoIV | KX574342 |
| Pike-perch iridovirus | PPIV | KX574341 |
| Lumpfish ranavirus isolate F140-16 | LMRV-F140-16 | MH665359 |
| Lumpfish ranavirus isolate F24-15 | LMRV-F24-15 | MH665358 |
| Lumpfish ranavirus isolate V4955 | LMRV-V4955 | MH665360 |
| Andrias davidianus ranavirus | ADRV-2010SX | KF033124 |
| Chinese giant salamander iridovirus | CGSIV-HN1104 | KF512820 |
| Common midwife toad virus | CMTV-Lv/2015 | MF004272 |
| Common midwife toad virus | CMTV-Pe/2015 | MF125269 |
| Common midwife toad virus | CMTV-Pe/2016 | MF125270 |
| Pelophylax esculentus virus | PEV | MF538627 |
| Rana catesbeiana virus isolate RC-Z | RCV-Z | MF187210 |
| Rana esculenta virus | REV | MF538628 |
| Trioceros melleri ranavirus 1 | TMRV1 | MG953519 |
| Trioceros melleri ranavirus 2 | TMRV2 | MG953520 |
| Terrapene carolina carolina ranavirus | TCCRV | MG953518 |
| Frog virus 3 | FV3-Op/2015 | MF360246 |
| Rana nigromaculata ranavirus isolate | RNRV- | MG791866 |
| MWH421017 | MWH421017 | |
| Zoo ranavirus isolate 040414 | ZRV | MK227779 |
| Wamena virus | WV | MT507284 |

1. A mammalian expression system comprising an attenuated, recombinant Ranavirus strain that has at least one expression element, wherein the attenuated, recombinant Ranavirus strain is Ambystoma tigrinum virus and further comprising at least one mammalian transcriptional element and at least one translational enhancement element that are incorporated into the Ranavirus strain.

2. The mammalian expression system of claim 1, wherein the at least one mammalian transcriptional element and the at least one translational enhancement element expresses a green fluorescent protein that is fused to a selectable marker, neomycin resistance (GNR) in non-permissive cells in vitro, in differentiated primary human cells ex vivo, and in mouse lungs and trachea in vivo without viral replication.

3. The mammalian expression system of claim 1, wherein the at least one mammalian transcriptional element and the at least one translational enhancement element comprises ATVΔ40L-SEL-GNR, ATVΔ40L-GFP or a combination thereof.

4. The mammalian expression system of claim 1, wherein the expression element expresses at least two proteins.

5. The mammalian expression system of claim 1, wherein the expression element expresses at least two proteins fused together.

6. The mammalian expression system of claim 1, wherein the system delivers antigens in mammalian cells without replication while generating antiviral immunity for mammalian respiratory diseases.

7. A method of delivering antigens to a mammal, comprising:
providing a mammalian expression system comprising an attenuated, recombinant Ranavirus strain that has at least one expression element; and
administering to the mammal a therapeutic amount of the attenuated, recombinant Ranavirus strain that has at least one expression element.

8. The method of claim 7, wherein the attenuated, recombinant Ranavirus strain is Ambystoma tigrinum virus.

9. The method of claim 7, further comprising at least one mammalian transcriptional element and at least one translational enhancement element that are incorporated into the Ranavirus strain.

10. The method of claim 9, wherein the at least one mammalian transcriptional element and the at least one translational enhancement element delivers GNR in non-permissive cells in vitro, in differentiated primary human cells ex vivo, and in mouse lungs and trachea in vivo without viral replication.

11. The method of claim 9, wherein the at least one mammalian transcriptional element and the at least one translational enhancement element comprises ATVΔ40L-SEL-GNR, ATVΔ40L-GFP or a combination thereof.

12. The method of claim 7, wherein the expression element expresses at least two proteins.

13. The method of claim 7, wherein the expression element expresses at least two proteins fused together.

14. A method of delivering antigens to a mammal to reduce the occurrence of mammalian respiratory disease, comprising:
providing a mammalian expression system comprising an attenuated, recombinant Ranavirus strain that has at least one expression element; and
administering to the mammal a therapeutic amount of the attenuated, recombinant Ranavirus strain that has at least one expression element.
